(19) United States
(12) Patent Application Publication
(10) Pub. No.: US 2010/0317730 A1
(43) Pub. Date: Dec. 16, 2010

(54) Treatment for menopausal and perimenopausal vasomotor symptons

(76) Inventor: Elias K. Shaya, Lutherville, MD (US)

Correspondence Address: Sam Rosen, Esquire;HODES, PESSIN & KATZ, P.A., Suite 400, 901 Dulaney Valley Road, Towson, MD, 21204, US

(21) Appl. No.: 12/802,651

(22) Filed: Jun. 11, 2010

Related U.S. Application Data
(60) Provisional application No. 61/186,535, filed on Jun. 12, 2009.

(51) Int. Cl. A61K 31/35; A61P 5/24

(52) U.S. Cl. 514/454

(57) ABSTRACT

Disclosed is a medicinal composition and method of use comprising topiramate for treating hot flashes and excessive sweating known as vasomotor symptoms related to perimenopausal or menopausal symptoms and in a still further embodiment of the invention topiramate and the medicine for treating menopause and perimenopause are provided in a kit or in unit dosage form, e.g., tablet or capsule. As a further embodiment of this invention, topiramate may be administered along with a medicament use to treat hot flashes and excessive sweating related to perimenopausal or menopausal symptoms.

### RELATED APPLICATIONS

This application is related to provisional patent application Ser. No. 61/186,535 filed Jun. 12, 2009.

It is also related to application attorney docket 027969.003 titled Hyperhidrosis Treatment, filed concurrently this day.

### FIELD OF THE INVENTION

The herein disclosed invention finds applicability in the field of medicine and for treating hot flashes and excessive sweating (hyperhidrosis) related to perimenopausal and menopausal symptoms. The present disclosure relates to improvements in the treatment of menopausal and perimenopausal hot flashes and hyperhidrosis, also known as vasomotor symptoms. Most particularly, the invention relates to delivery of a pharmaceutically acceptable amount of a drug, or prodrug or metabolite thereof.

### BACKGROUND OF THE INVENTION

An article entitled “Advances in the Treatment of Menopausal Symptoms” published in July 2009 as a continuing medical education activity on cme.medscape.com provides a comprehensive review of the literature and represents the current standard of medical practice in the treatment of menopausal symptoms (ref., JoAnn Pinkerton et al). As noted in said article, the terms “hot flashes”, “hot flushes”, “night sweats”, “climacteric symptoms”, “vasomotor symptoms”, and “VMS” are often used interchangeably.

Perimenopausal or menopausal vasomotor symptoms affect 75% of menopausal women (estimates range between 68% and 90%). Such symptoms cause severe distress and suffering in some patients and may also impair sleep, especially in patients with night sweats.

Currently there is no approved treatment for these symptoms except hormone therapy. However, two major studies, the HERS and WHI studies were published in 2002 and reported serious concerns about severe and dangerous side effects caused by hormone therapy that included heart disease and increased risk of certain cancers, amongst others. Prior to the release of the HERS and WHI study results, approximately 42% of women aged 50-74 years were taking hormone therapy for help with these symptoms. Following the publication of HERS and WHI results, there has been a substantial drop in the use of hormone therapy and no other treatment has been approved since. WHI study Rossouw et al (2002), JAMA Vol. 288 (3) pages 321-33. HERS study Hulley et al (August 1998), JAMA Vol. 280 (7) pages 605-13.

Hot flashes and excessive sweating often cause great emotional distress and occupational disability for the patient. These patients experience poor sleep and other negative effects on their quality of life. They frequently have to get up in the middle of the night to change their wet clothes or even to change their bedding at times. They report feeling embarrassed at work and their condition becomes acutely worse if they are under stress, and they feel more self conscious if they have to present at meetings, as their shirts and clothes may appear evidently wet.

Currently, the only approved treatment for vasomotor symptoms of menopause and perimenopause is hormone replacement therapy. Other off label and unapproved treatments that have demonstrated some modest benefits are limited by significant adverse effects. These may include anticholinergics and clonidine, as well as selective serotonin reuptake inhibitors (paroxetine, fluoxetine, and venlafaxine). Some life style changes, environmental manipulation, and herbal therapies have been tried and some have shown slight superiority over placebo. However, these studies had some methodological shortcomings and included small numbers.

Despite the high prevalence of perimenopause and menopause and despite the great need of a large segment of this population for and alternative to hormone therapy, no other treatment has been approved. Even after almost two decades of availability of topiramate and the long felt need in the market for an effective treatment for vasomotor symptoms of perimenopause and menopause, no other person has observed topiramate as a remedy for these symptoms. This included the pharmaceutical company and its scientists who had a close and intimate understanding of this drug and its various characteristics and who first patented and marketed topiramate under the brand name of Topomax for other indications but never noted its efficacy in the treatment of the vasomotor symptoms of menopause. Furthermore, these scientists and others with intimate knowledge of topiramate did not note the efficacy of topiramate in the treatment of vasomotor symptoms of menopause even though one of the rare but listed side effects of topiramate is decreased sweating (hypohidrosis). In the context of its use for the treatment of seizures especially in children. Furthermore, those and other scientists must be aware of published case reports that observed efficacy of topiramate in the treatment of primary palmar hyperhidrosis. Nevertheless, to the best of the inventor's knowledge, no one has made an observation of the efficacy of topiramate in the treatment of menopausal or perimenopausal vasomotor symptoms. This is despite the great need for some remedy for this condition and the significant number of women who could benefit from this treatment.

### Review of Literature

An extensive review of the literature performed by the inventor as well as the comprehensive review article mentioned above revealed no prior mention of the use of topiramate for perimenopausal or menopausal vasomotor symptoms.

### OBJECTS OF THE INVENTION

An object of this invention is to produce a therapy which is safe and effective for treating hot flashes and excessive sweating associated with menopause and perimenopause.

A further object of this invention is to provide a therapy useful for treating vasomotor symptoms associated with menopause and perimenopause.

A still further object of this invention is to provide a pharmaceutical composition containing an ingredient for treating symptoms of menopause or perimenopause along with a component for preventing vasomotor symptoms, hot flashes and excessive sweating.

These and other object of the present invention will become apparent from a reading of the following specification taken in conjunction with the claims.

### BRIEF SUMMARY OF THE INVENTION

Based on data and observations made by the inventor, he has found topiramate to have the following, previously undiscovered, beneficial pharmacological effects for patients with excessive sweating and hot flashes. They are as follows:
1. Substantial improvement in vasomotor symptoms, excessive sweating and hot flashes in perimenopausal and menopausal patients.
2. Helps to prevent patients from experiencing hot flashes, excessive sweating, and vasomotor symptoms associated with their perimenopause and menopause.
3. Substantial improve or completely suppress nighttime vasomotor symptoms, excessive sweating, and hot flashes associated with perimenopause and menopause.
4. Substantial improve or completely suppress daytime vasomotor symptoms, excessive sweating, and hot flashes associated with perimenopause and menopause.
5. Effective in controlling these symptoms when taken only on an as needed basis.

The patients in whom the inventor has seen these benefits with topiramate experienced minimal to no side effects due to the use of small doses of this drug. The patients' quality of life was substantially improved as they no longer had to change their night clothes or bedding in the middle of the night and they were no longer embarrassed and self conscious at work and at meetings where they would otherwise be sweating profusely while others in the same environment appeared to be quite comfortable.

Hot flashes and excessive sweating are quite uncomfortable and even embarrassing to patients. Thus, a suitable treatment for these patients is highly desirable, especially in that at the present time there are no other approved and safe treatments available.

The herein disclosed invention describes a method of treating hot flashes and hyperhidrosis caused by perimenopause and menopause.

### DESCRIPTION OF THE INVENTION

Topiramate is currently available in 25 mg and 100 mg tablets, and is approved for the treatment and prevention of epilepsy and migraine headaches in doses ranging from 100 mg to over 400 mg a day.

According to the present invention, topiramate doses as low as 12.5 mg per day have been found to be highly effective in treating hot flashes and hyperhidrosis in perimenopausal and menopausal women. Optionally, the topiramate can be taken as a low dose on an as-needed basis. Furthermore, while in higher doses, topiramate is known to have potentially undesirable side effects, the low dosage found to be effective in embodiments of the present invention is able to provide effective results without significant incidence of those side effects.

In preferred embodiments of the invention, the topiramate could be provided in a relatively low dosage of 12.5 mg or lower, or in higher doses if necessary. It may be formulated in an extended release, skin patch, inhaled nasal applicator, or other formulations. In these formulations, topiramate, may be the only active ingredient or as part of a combination that includes other active or inactive ingredients. Further, for patients diagnosed with night sweats, it may be preferable to use topiramate at night only.

Herein listed are drugs useful for treating menopausal and perimenopausal vasomotor symptoms which may be used in combination with topiramate in order to impede vasomotor symptoms, hot flashes and excessive sweating; such medicaments are listed below and other drugs known in the art are not herein mentioned:
Oral Estrogen for hot flashes, vulvar and vaginal atrophy, taken in usual doses of 0.625 mg daily or smaller or potentially higher doses if necessary.
Topical estrogen (for vaginal atrophy, important because it's commonly used in low doses and is not systemic, so it's qualitatively different from other preparations of estrogen and is effective for local vaginal symptoms and where the combination with topiramate helps the systemic vasomotor symptoms).
MF-101 (an estrogen receptor beta agonist for treatment of vasomotor symptoms).
Centrally acting antidepressants, gabapentin, clonidine (oral or transdermal), venlafaxine, desvenlafaxine, paroxetine, and progesterone (all non-hormonal treatments for vasomotor symptoms. Various lower doses of these medications may then be used when combined with topiramate with the expectation of improved benefits for the patient and fewer side effects.

Typical compositions of medicament and topiramate:
Progestin (for hot flashes, vulvar and vaginal atrophy, and prevention of osteoporosis).
Tamoxifen, with or without estrogen (for prevention of breast cancer).
Raloxifene, with or without estrogen (for prevention and treatment of osteoporosis, and reduction of risk of invasive breast cancer in postmenopausal women with osteoporosis and a high risk of invasive breast cancer).
Bazedoxifene, with or without estrogen (for prevention of osteoporosis).
These combinations of medications may be used with topiramate in low doses of 5-15 mg.

Higher doses of topiramate may be used if necessary. The medications listed for combination with topiramate may also b used in smaller than their usual doses with the advantage of fewer and less intense side effects.

Tablet

| | Estrogen | conventional dose |
|---|---|---|
| Dose | Topiramate | 12.5 mg |

In a tablet carrier.

### Kit

Dose Progestin tablet conventional dose
   Topiramate tablet 25 mg
Both in a glassine envelope.

Said another way the disclosed invention involves a method for the treatment of perimenopausal and menopausal vasomotor symptoms, hot flashes, and excessive sweating, comprising the administration to a patient an effective amount of topiramate, wherein the effective amount of topiramate administered is from about 12.5 mg per day or higher doses if necessary to achieve the desired effects. The topiramate can be administered in an extended release formulation, by mouth, as a skin patch, ointment or nasal spray. The method of this invention can be practiced using a kit containing medication comprising as a first component a medicament which is useful for treating menopause or perimenopause and as a second component topiramate to impede vasomotor symptoms, hot flashes and excessive sweating or can be used as a composition in unit dosage form comprising an effective amount of topiramate and a medicament for treating menopause or perimenopause wherein the composition impedes vasomotor symptoms, hot flashes and excessive sweating.

### Example of Clinical Use

Mrs. H had been stable on her current treatment for Panic and Major Depressive Disorders with a satisfactory remission for several years and without any side effect complaints. However, now at the age of 55, she started developing menopausal symptoms of hot flashes and excessive sweating. After counselling and informed consent about the off-label use, she took a trial of topiramate 25 mg by mouth twice daily. Upon follow up, she confirmed that the excessive sweating was improved with this low dose of topiramate and that she has found it to be effective when taken on an as needed basis, depending on her expectations of physical activity and weather/temperature conditions. She reported no adverse effects with the use of topiramate. Several other similar clinical examples were observed.

Obviously, many modifications may be made without departing from the basic spirit of the present invention. Accordingly, it will be appreciated by those skilled in the art that within the scope of the appended claims, the invention may be practiced other than has been specifically described herein.

What is claimed is:

1. A method for the treatment of perimenopausal and menopausal vasomotor symptoms, hot flashes, and excessive sweating, comprising the administration to a patient an effective amount of topiramate.

2. The method of claim 1, wherein the effective amount of topiramate administered is from 12.5 mg or less per day or higher doses if necessary to achieve the desired effects.

3. The method of claim 2, wherein the topiramate is administered to an extended release formulation, by mouth, as a skin patch, ointment or nasal spray.

4. The method of claim 2, wherein the topiramate is administered at night.

5. A kit containing medication comprising as a first component a medicament which is useful for treating menopause or perimenopause and as a second component topiramate to impede sweating.

6. A composition in unit dosage form comprising an effective amount of topiramate and a sweat indulging medicament for treating menopause or perimenopause.
